(19) United States
(12) Patent Application Publication
(10) Pub. No.: US 2025/0104449 A1
(43) Pub. Date: Mar. 27, 2025

(54) METHOD FOR DETECTING AT LEAST ONE MEDICAL CONSUMABLE

(71) Applicant: Clinomic Medical GmbH, Aachen (DE)

(72) Inventors: Arne PEINE, Aachen (DE); Lukas MARTIN, Aachen (DE)

(21) Appl. No.: 18/294,744

(22) PCT Filed: Aug. 4, 2022
(86) PCT No.: PCT/EP2022/000070

§ 371 Date: Feb. 2, 2024

(30) Foreign Application Priority Data
Aug. 5, 2021 (DE) 10 2021 120 443.7

(51) Int. Cl. G06V 20/64; G06V 10/764; G06V 10/774; G06V 10/82; G10L 13/02

(52) U.S. Cl. CPC G06V 20/64; G06V 10/764; G06V 10/774; G06V 10/82; G10L 13/02; G06V 2201/03

(57) ABSTRACT

A method for acquiring at least one medical consumable is provided. The method comprises conducting a provision of image information by an image recording device via at least the one medical consumable, whereby the image information is specific for a form of the medical consumable; conducting an application of an image evaluation means for shape recognition of the shape of the medical consumable, with the provided image information as input to the image evaluation means, in order to use an output of the image evaluation means as classification information about the medical consumable; and generating audio information based on the classification information to initiate an acoustic output of the audio information via an audio device.

The present invention relates to a method for acquiring at least one medical consumable. The invention further relates to a system and a computer programme.

It is known from the prior art, for example from publications CN 101 727 651 A and JP 2005 309 702 A, that medical consumables can be recognised by means of a computer-readable identification feature in order to provide monitoring of the consumption of disposable material. Publication U.S. Pat. No. 10,831,865B2 also discloses a method of recognising medication by means of a camera, whereby information about the medication is displayed on a screen.

One of the disadvantages of the known solutions is that the number of consumables in the hospital and especially in acute medicine are not recorded per patient. There may therefore be no opportunity for further evaluation, for example to determine how many consumables are used for a particular disorder. Furthermore, a display on a screen is disadvantageous in certain situations, for example when the operator has to interrupt his regular workflow and focus on the screen. Particularly in poor lighting conditions or due to reflections, for example from lamps, the use of a screen in a medical environment can lead to impairment of activity.

It is therefore an object of the present invention to at least partially eliminate the disadvantages described above. In particular, one objective is to improve the recording of medical consumables.

The above mentioned function is solved by a method with the features of claim 1, a system with the features of claim 21 and by a computer programme with the features of claim 28. Further features and details of the invention can be seen from the respective sub-claims, the description and the drawings. Features and details described in connection with the method according to the invention naturally also apply in connection with the system used in accordance with the invention and the computer programme used in accordance with the invention, and vice versa in each case, so that reference is or can always be made to the individual aspects of the invention with respect to the disclosure.

The problem is solved in particular by a method for recording, in particular goods recording, of at least one medical consumable. Here the intention is that the following steps are carried out, whereby the steps are preferably carried out one after the other in the specified sequence or in any sequence, and individual steps can also be carried out repeatedly:
rendering of an image recording device, in particular with at least one image sensor, preferably in the form of a camera,
making available image information about at least one medical consumable by the image recording device, the image information preferably being specific to a form of the medical consumable,
conducting an application of an image analysing means, in particular for shape recognition of the shape of the medical consumable, with the provided image information as an input of the image analysing means to use an output of the image analysing means as a classification information about the medical consumable, whereby advantageously the image analysing means generates the output based on the input, whereby the application of the image analysing means for this purpose preferably comprises the execution of an algorithm or a neural network, e.g. for the shape recognition,
generating audio information based on the classification information in order to initiate an acoustic output of the audio information via an audio device, in particular by means of speech synthesis for speech output of the classification information.

The following terminology should also be explained here:
In the context of the invention, indefinite and definite articles or numerals, e.g. “one”, “two”, “etc.”, are always to be understood as “at least” indications, unless expressly stated otherwise. Moreover, numerical data and data on process parameters and/or device parameters are to be understood in the technical sense, i.e. as being provided with the usual tolerances. Even from the explicit specification of “leastwise” or “at least” or similar, it should not be concluded that the simple use of the article or the number, i.e. without specifying “at least” or similar, a restriction, e.g. in the sense of “precisely”, should be implied. The intention is specifically, that the image recording device may also be a number of image recording devices, the image information may also be a number of image information devices, the consuming means may also be a number of consuming means, the image analysing means may also be a number of image analysing means, the audio processing device may also be a number of audio processing devices and the audio information may also be a number of audio information devices.

A medical consumable is understood in particular to be a medical consumable, for example catheters, scalpels, needles, swabs or spatulas, whereby the list is not exhaustive. The consumables covered by the method according to the invention may comprise different types of consumables, for example at least two or at least three or at least four of the following types of consumables: catheters, scalpels, needles, swabs, spatulas, bandages, plasters, medication, infusion solutions, syringes, cannulas, gloves, gowns, aprons, face masks, head caps, kidney bowls, wash bowls, bedpans, urine bottles, dispensers. In other words, the method according to the invention may not only detect one of the types of consumables, but may be suitable for detecting different types. Here, the method according to the invention can be carried out to distinguish between the different types of consumables and to indicate them via the classification information, preferably by using the image evaluation means.

An image recording device describes in particular a device which is designed to provide at least one image or video recording, whereby the recording can be initiated manually by an operator or automatically by a trigger or automatically at regular intervals. The image recording device can be, for example, a camera. Image information is understood to mean, in particular, an image or video recording, preferably in digital form. A “shape of the medical consumable” refers in particular to an external plastic form with specific contours. This shape can be present in the image or video recording and can be identified in particular by the shape recognition, e.g. by means of pattern recognition.

The use of an image evaluation means is understood here in particular to mean that image processing is first carried out in order to analyse image information, for example a photograph or at least or exactly one individual image from a video, in relation to certain features, for example in relation to the shape. For example, at least one algorithm such as edge and/or pattern recognition can be used for this purpose. Moreover, in the context of the present invention, the medical consumable corresponding to the shape is recognised and assigned by the image evaluation means on the basis of this analysis. Information assigned to the respective medical consumable, for example a designation of the medical consumable such as “spatula” or a batch number, is preferably referred to here as the classification information. The image evaluation means can be designed, for example, as an algorithm or as a neural network.

The use of the image evaluation means makes it possible, for example, to recognise the type and/or the product class of the medical consumable based on the image information and release it as the classification information. The type or class of goods may include at least one or at least two or at least three or at least four of the following: catheters, scalpels, needles, swabs, spatulas, bandages, plasters, medication, infusion solutions, syringes, cannulas, gloves, gowns, aprons, face masks, head caps, kidney bowls, wash bowls, bedpans, urine bottles, dispensers. The recognition can be based on the shape, with alternative or additional features of the consumable such as a colour or a size or another pattern also being used. The application of the image evaluation means is preferably not understood as the recognition of coded information such as a barcode.

Audio information is understood to mean, in particular, information that makes the classification information suitable for acoustic release, so that it can be released via the audio device. For example, speech synthesis can be used to transform a text into a synthetic language.

According to the invention, the image recording device and/or the audio device can be part of a system.

In particular, the acoustic output of the audio information does not merely comprise a voice output of a graphical user interface, i.e. preferably the voice output of the text information of the user interface, but rather the dedicated output of the classification information. It may also be possible that a system according to the invention does not provide a graphical user interface and/or the classification information is not released via a graphical user interface, but only as an audio output.

In addition, it is optionally provided that the method further includes:
provision of a recording area, in particular a support surface, in particular of the system according to the invention and/or arranged on a housing of the system, preferably in order to place the at least one medical consumable thereon and/or hold it thereover, whereby the recording area or the support surface can be transparent in order to enable recording of the medical consumable by the image recording device through the recording area or the support surface,
conducting the recording of the image information by the image recording device, the recording being initiated electronically and/or manually by an operator or automatically at regular intervals to provide the image information.

In simple terms, a surface can be provided here on which the operator places the at least one medical consumable in order to enable the recording of at least the one medical consumable. thereby it can be envisaged that the operator actively e.g. by pressing a button, initiate the recording or that the recording is automatically initiated by means of a pressure sensor due to the weight of the medical consumable. Furthermore, it may be possible that, in the case of automatic initiation at regular intervals, the regular intervals are less than 2 seconds in order to prevent at least the one medical consumable from being applied too briefly and thus to prevent incorrect recording of at least the one medical consumable or at least to reduce the probability thereof. Furthermore, it is conceivable that the recorded image information includes a plurality of image information, and thus corresponds to video information, in order to then use individual images of the video information as the input for the image evaluation means. Provision of a support surface can have the advantage that the operator can carry out his regular activity while the method for detecting at least the medical consumable is being carried out. Particularly in the case of a longer recording period, it is advantageous if the operator does not have to hold the consumable above or on the image recording device for the duration of the recording. Alternatively, the recording area can also be arranged and/or designed in such a way that it is not possible to place it on the recording area and therefore the consumable must be held in front of it. Conceivable, for example, is a vertical orientation of the recording area. This enables flexibility to the attachment, e.g. on a wall of a hospital room. Furthermore, it may be possible for the housing to have at least one fastening means in order to be able to mount the recording area in both vertical and horizontal orientation. The horizontal alignment can enable the recording area to be used as a support surface. The fastening element is a drill hole, for example, or an adhesive or the like.

In addition, within the scope of the invention it is optionally conceivable that the method further comprises:
performing a recording of the image information by the image recording device, the medical consumable for recording being held over the image recording device by the operator, the recording being initiated electronically and/or manually by the operator or automatically at regular intervals to provide the image information.

The described directional indication that the medical consumable is held by the operator above the image recording device is to be understood in particular as meaning that the medical consumable is thereby located parallel to the direction of gravity above the image recording device. Holding it over the image recording device has the advantage of enabling rapid recording of even several consumables one after the other. The duration of exactly one single recording of the image information is advantageously less than 2 s (s stands for second).

Another advantageous aspect of the invention is that the method further comprises:
Conducting speech synthesis to provide an artificial generation of a human speaking voice from the audio information to acoustically output the audio information based on the human speaking voice via the audio device, preferably to verbally communicate the classification information to the operator, preferably to communicate the class of goods and/or the type of consumable.

In other words, a text such as continuous text is converted into an acoustic speech output. It may be possible for voice recordings to be used for this purpose, which comprise the designation of at least the one medical consumable and/or further information relating to the at least one medical consumable. Furthermore, it may be possible that a supplementary text-to-speech system is applied.

According to an advantage of the invention, the method may further comprise:
provision of an interface, in particular of the system according to the invention, for communication with a merchandise management system and/or a patient data management system,
transmitting at least partial classification information and/or information resulting therefrom to the merchandise management system and/or to the patient data management system.

Communication can be provided, for example, via a WLAN, LAN, or Bluetooth interface, where WLAN stands for wireless local area network and LAN stands for local area network. The merchandise management system and/or the patient data management system can optionally be provided by an external data processing system, preferably centrally for several computers in a hospital. Transmitting the classification information can have the advantage that the consumption of medical consumables per patient and consequently per disorder can be determined centrally. This allows for more precise advance planning of medical consumables.

An advantageous aspect of the invention is that the method further comprises:
transmission of at least one identification characteristic of the patient, in particular a name and/or a date of birth, by the patient data management system to the image analysis means in order to subsequently add the at least one identification characteristic to the audio information in order to verify an allocation of at least the one medical consumable to the correct patient.

In other words, in particular each time a medical consumable is acquired, an identification feature, for example the patient's name, is issued to enable the operator to check whether this is the correct allocation of the patient for whom the medical consumable was used. This can have the advantage of preventing technical errors or recognising them early.

It is also conceivable that an input option, preferably, according to the invention, of the system becomes available which enables input by an operator. The input can, for example, be a voice input and/or activate a function to correct and/or delete incorrect classification information and/or patient allotment.

Furthermore, it can be provided within the scope of the invention that the image evaluation means is designed as a neural network, in particular as a neural convolution network. The neural convolutional network can be a so-called Convolutional Neural Network (CNN). In a further possibility, it is possible that the method further comprises:
conducting training of the image evaluation means, in particular before carrying out the application of the image evaluation means,
   whereby preferably the image evaluation means is trained and validated using training data, in particular a proportion of more than 80%, particularly preferably 908, of the training data being used for training and the remaining proportion of the training data being used for validation, whereby the training data contain at least one piece of image information of at least one medical consumable.

The image evaluation means is in particular a neural network in order to modify the weights of the individual neurons of the neural network during training, preferably on the basis of the training data. The training can in particular involve supervised learning, in which the correct, at least one medical consumable is specified in the training data in order to use this to optimise the weightings of the neural network. To put it simply, the neural network tries to derive patterns and laws and accordingly adapt to the patterns and laws. In particular, validation involves using training data that is still unknown to the neural network to determine whether the neural network recognises the correct, and, at least one medical consumable.

It should be noted that an image evaluation means which was trained by the training, described above, is also the subject of the invention. This image evaluation means has at least one training result, e.g. a classifier or a weighting that is specific to the training carried out and the training data used.

Furthermore, within the scope of the invention it is optionally conceivable that for the execution of the training to further comprise:
acquiring training image information, in particular by an image recording device,
conducting a detection of an image of at least one medical consumable in the acquired training image information, whereby the acquired training image information is used as training data if the detection indicates a presence of the image, and otherwise the acquired training image information is used as a reference for the training.

In particular, the reference is understood to mean image information in which no medical consumable is present, in order to subsequently be able to derive a difference from image information in which at least one medical consumable is present on the basis of the reference, in which the difference corresponds to at least one medical consumable present, in particular in order to record at least one medical consumable, in particular its shape. It is also possible that training using the reference is carried out again subsequently (i.e. after training), e.g. in order to adapt the procedure for different environments. In doing so, it may be possible for the operator to initiate this renewed execution of the partial training.

Furthermore, it is advantageous if, in the context of the invention, carrying out the training further comprises:
Acquiring a confirmation of the classification information of the image analyser based on the output of the audio information via the audio device by an operator.

The confirmation by the operator can have the advantage that the image evaluation device receives feedback as to whether at least one medical consumable was correctly recorded, in order to enable the image evaluation device to be adjusted in the event of incorrect detection. The confirmation can e.g. be done by an input such as a voice input.

Furthermore, it is optionally envisaged that the training data comprise at least one piece of training image information and that a brightness of at least one piece of training image information varies in order to enable the image evaluation means to be used when the brightness of the at least one piece of image information varies. The use of a variation in the brightness of at least one piece of training image information has the particular advantage that it can be used in a poorly lit environment, for example a hospital environment.

Furthermore, it may be possible within the scope of the invention that the training data includes at least one piece of training image information in which the medical consumable to be acquired is partially obscured. A partial concealment of the medical consumable in at least one piece of training image information can have the advantage that it imitates contamination, for example by blood, or an actual concealment of the medical consumable.

Furthermore, it may be possible within the scope of the invention that the training data includes at least one piece of training image information in which at least two medical consumables to be recorded are present. This can have the advantage that at least two medical consumables can be recorded at once, which means that overall less time is required for the recording.

In a further possibility, it is possible that the method further comprises:
Making available an external database and an external data processing device in order to perform the image analysis.

The term “external” refers to the fact that the database and the data processing device are present as a separate, stand-alone or connected system and can also be used independently of the recording of at least the one medical consumable. Optionally, “external” also means “outside the system pursuant to the invention”. It is particularly conceivable that the database and the data processing device are present centrally in a hospital in order to provide and update the classification information about medical consumables present in the database centrally for the entire hospital.

It may further be possible for the external database to provide at least the one classification information of at least the one medical consumable, wherein the classification information of at least the one medical consumable is assigned to at least one form.

According to an advantage of the invention, the method further comprises:
transmitting the respective image information acquired by the image recording device to the external data processing device in order to subsequently determine the classification information of at least the one medical consumable available in the external database and associated with the acquired image information, whereby the image information can thereby be specific to a shape of the medical consumable, that the image information comprises an image of the shape of the medical consumable in order to subsequently use the classification information corresponding to the shape to generate the audio information if the image information associated with the respective classification information in the external database correlates with the imaged shape in the range of a defined order of magnitude.

To put it simply, here in particular the image information is transmitted by the image recording device to the external data processing device in order to compare the form of at least the one medical consumable in the transmitted image information with at least the one form of a medical consumable in the database. During the comparison, it is checked in particular whether the shape of the recording of at least the one medical consumable correlates within a specified range with the shape of a medical consumable present in the database. The classification information corresponding to the shape can then be used to generate the audio information. This can have the advantage that only the external data processing device needs to have high computing power, while the stationary device, for example in a hospital room, is only intended for image recording, the generation of audio information and its acoustic output.

Furthermore, it is optionally provided that the method further comprises:
updating the database, the updating being carried out regularly by transmitting training data, the training data being created or received as part of a training described above.

This can have the advantage that the data in the database is optimised. It may be possible that when a new medical consumable is added, the database is supplemented with this.

It is also possible for at least one of the following steps to be carried out in the method according to the invention:
recording information about a patient for whom the recorded consumable is used and/or to whom the consumable is allocated in the database, the information preferably comprising a name and/or a date of birth of the patient,
recording information about a clinical picture (of the patient) to which the consumable is allocated in the database and/or for whose treatment the recorded consumable is used,
recording information about a number of consumables which are recorded and/or are allocated to the consumable in the database and/or are to be used,
acquiring at least one of the aforementioned information through a user input, in particular a voice input, and/or by reading out a digitally, non-volatilely stored pre-setting,
allocating at least one of the aforementioned pieces of information to the medical consumable, preferably in the database, preferably by linking the classification information with at least the one aforementioned piece of information using information technology,
Generating the audio information additionally on the basis of at least one of the aforementioned pieces of information, whereby the audio information is generated in particular on the basis of the information about the patient and/or the clinical picture and/or the number, in order to acoustically output the name of the patient and/or the date of birth and/or the clinical picture and/or the number together with the classification information, preferably the type of consumable.

This has the advantage that a user can acoustically check whether the type of consumable has been correctly identified and assigned to the correct patient and/or clinical picture and/or the correct number.

It may be possible for the medical consumable to be detected in that the classification information, preferably associated with information about a patient and/or a clinical picture (of the patient) and/or a number, is stored in a non-volatile manner and/or output acoustically and/or stored in a database such as a goods registration system. In contrast to the pure identification of a consumable, recording in the sense of registering goods implies that the classification information is retained. It is possible for a link and/or communication to be established between a goods registration system and a patient data management system in order to allocate the information about the patient and/or the clinical picture to the classification information.

Furthermore, according to an advantage of the invention, the method further comprises:
A storage of at least the one recorded medical consumable associated with a clinical picture and/or a patient in order to enable individual recording of at least one required medical consumable per patient and/or per clinical picture.

It may be possible that the recording per clinical picture includes further information about the individual course of the clinical picture, for example a degree of severity or whether complications have occurred, in order to enable even more precise planning of the medical consumables.

It may further be possible that the classification information comprises a designation of the medical consumable to enable a check whether the at least one medical consumable has been correctly detected, whereby for the check an operator compares the audio information output via the audio device with the at least one detected medical consumable.

A designation is understood in other words to mean in particular the type of medical consumable, for example “spatula”. Furthermore, it can be provided that the name includes further characteristic information about the medical consumable, for example the manufacturer.

The classification information may preferably also include a batch number of the at least one medical consumable.

Recording the batch number can have the advantage of providing traceability of the medical consumables, so that, on the one hand, it can be determined which products a specific patient has received and, on the other hand, to which patients a specific batch was administered.

The invention also relates to a system for detecting at least one medical consumable, in particular consumables, comprising at least:
an image recording device, in particular an electronic image recording device, for providing at least one piece of image information about at least the one medical consumable, whereby preferably the image information is specific for a form of the medical consumable,
an image processing device for conducting an application of an image evaluation means, in particular for shape recognition of the shape of the medical consumable, with the provided image information as an input of the image evaluation means, in order to use an output of the image evaluation means as classification information about the medical consumable,
an audio processing device for generating audio information based on the classification information,
an audio device to perform an acoustic output of the audio information. As a result, the system according to the invention has the same advantages as those described in detail with reference to a method according to the invention.

In addition, the system can be suitable to conduct the method pursuant to the invention. The system according to the invention can also be designed as a medical device, in particular as a stationary device. The device can also be designed as a “bedside” device for placement and/or attachment adjacent to a patient bed.

In a further option, the system can be designed as a stand-alone system, whereby in particular a single, common housing is provided for all components of the system. The housing can be designed to be disinfectable in order to enable the system to be provided, for example, in an intensive care unit in a hospital.

Stand-alone means in particular that the system can fulfil its function without additional devices and is specifically designed as a modular construction. However, the system according to the invention can of course have interfaces for data exchange and/or for energy supply and/or for modular expansion. For modular expansion, for example, an external audio device can be connected through one of the interfaces.

It may optionally be possible for the image recording device to have a camera, in particular the image recording device having at least one floor element and/or frame in order to be set up in a room, in particular a hospital room, in such a way that the camera is aligned towards a ceiling of the room.

Here, it may be provided that the at least one floor element and/or frame provide a level of the system that allows standing operation by an average-sized operator. This can have the advantage that the operator has to interrupt his regular activity, which is largely carried out while standing, for a shorter period of time.

It is also advantageous if, within the scope of the invention, the image recording device has at least one lamp, in particular an infrared lamp, in order to illuminate a viewing area of the image recording device.

This can have the advantage that there is a predominantly constant and, above all, sufficient brightness of the image information recorded by the image recording device.

A further advantage can be achieved within the scope of the invention if the system has an interface for a data connection with a merchandise management system and/or a patient data management system, the interface being arranged in the housing.

The interface can be, for example, a Bluetooth, a WLAN or a LAN interface, although the list is not exhaustive. WLAN stands for Wireless Local Area Network and LAN stands for Local Area Network.

Furthermore, it may be possible within the scope of the invention that the system has a water-repellent and/or water-impermeable coating in order to be designed for use in a medical environment.

It is particularly conceivable that only parts of the system have a water-repellent and/or water-impermeable coating, for example the image recording device and/or the audio device.

The coating can have the advantage of protecting the electronics in the devices from damage caused by liquid.

Another object of the invention is a computer programme comprising instructions which, when the computer programme is executed by a computer, cause the computer to execute the method according to the invention. The computer program according to the invention thus brings with it the same advantages as have been described in detail with reference to the method according to the invention and the system according to the invention.

Further advantages, features and details of the invention emerge from the following description, in which exemplary embodiments of the invention are described in detail with reference to the drawings. In this respect, the features mentioned in the claims and in the description can each be essential to the invention individually or in any combination. It shows:

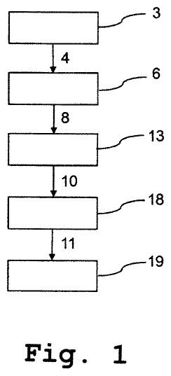

FIG. 1 Example of a schematic sequence of the process according to the invention

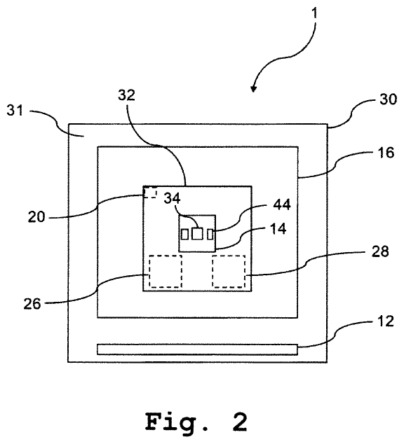

FIG. 2A top view of an embodiment of the system according to the invention

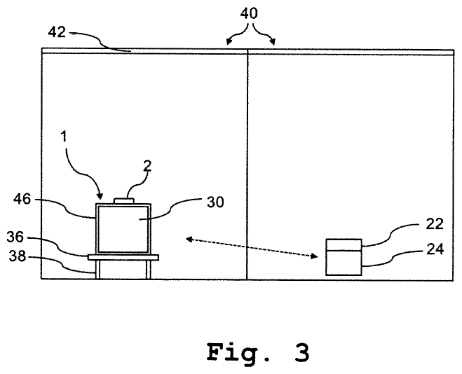

FIG. 3 Another embodiment of the system according to the invention with a data processing device and a database

FIG. 1 uses a schematic diagram to show a possible sequence of a method according to the invention. In the first step, the method begins with image recording 3 in order to subsequently provide the image information 4 to the image evaluation means 6. In the second step, the image information 4 is analysed by the image analysis means 6 in order to obtain the classification information 8 as output. In the next step, the audio information 13 is generated from the classification information 8, whereby the generated audio information 10 is then transferred to the speech synthesis 18. The audio information 10 can comprise part or all of the classification information 8. It is particularly conceivable that the audio information 10 only provides a comparison that can be carried out by an operator with regard to a patient and a medical consumable 2, while the remaining part of the classification information 8 is further processed, transmitted or stored in an optional step following the described sequence. The subsequent speech synthesis step 18 generates a synthesised speaking voice 11 from the audio information 10 in order to subsequently provide the speech output 19 via the audio device 12.

FIG. 2 shows a schematic top view of an embodiment of a system 1 according to the invention. The system 1 is arranged in a housing 30 and features an audio device 12 and a recording area 16, in particular in the form of a support surface 16, on a cover surface 31, whereby the recording area 16 or the support surface 16 can be designed to be transparent. The remaining elements according to the invention are visible through the recording area 16 or the support surface 16. An image recording device 14 is shown in the centre, which has a camera 34 and two lights 44 to the right and left of the camera 34. Viewed from this perspective, a processing housing 32 is shown behind the image recording device 14, in which an interface 20, an image processing device 26 and an audio processing device 28 are arranged.

FIG. 3 shows another embodiment of the system 1 according to the invention with a data processing device 24 and a database 22. What is particularly characteristic of this embodiment is that an image evaluation according to the invention can be carried out here by the externally located data processing device 24 and database 22. In this FIG. 3, the external location corresponds to an arrangement in a further room 40, whereby it is also conceivable that the data processing device 24 and the database 22 are arranged in the same room 40 as the system 1. The system 1 is arranged in a room 40, which features a ceiling 42. The system 1 includes a frame 38 and a floor element 36 on which the system 1 is placed. The system 1 further comprises a housing 30 which features a coating 46. A medical consumable 2 is stored on the system 1. Furthermore, a second room 40 is visualised, in which a data processing device 24 and a database 22 are arranged. The dotted line shows the bidirectional communication between the system 1 and the data processing device 24. The bidirectional communication is conceivable here in particular via a radio connection, although an electrical connection, such as a USB or LAN connection, may also be possible.

The above explanation of the embodiments describes the present invention solely in terms of examples. Of course, individual features of the embodiments can, if technically meaningful, be freely combined with one another without departing from the scope of the present invention.

### REFERENCE NUMBERS

1 system
2 medical consumables
3 image recording
4 image information
6 image evaluation means
8 classification information
10 audio information
11 speaking voice
12 audio device
13 generation of audio information
14 image recording device
16 recording area, support surface
18 speech synthesis
19 voice output
20 interface
22 database
24 data processing device
26 image processing device
28 audio processing device
30 housing
31 cover surface
32 processing housings
34 camera
36 floor element
38 frame
40 room
42 ceiling
44 light
46 coating

1. Method for detecting at least one medical consumable, where the following steps must be carried out:
provision of an image recording device,
performing a provision of image information about at least the one medical consumable by the image recording device, whereby the image information is specific to a shape of the medical consumable,
performing an application of an image evaluation means, for shape recognition of the shape of the medical consumable, with the provided image information as an input of the image evaluation means to use an output of the image analysing means as a classification information about the medical consumable,
generating audio information based on the classification information in order to initiate an acoustic output of the audio information via an audio device.

2. The method according to claim 1, wherein the method further comprises:
provision of a recording area, in order to place at least the one medical consumable thereon, the recording area being transparent in order to enable recording of the medical consumable by the image recording device through the recording area,
conducting the recording of the image information by the image recording device, the recording being initiated electronically and/or manually by an operator or automatically at regular intervals in order to provide the image information.

3. Method according to claim 1, wherein the procedure further comprises:
performing of a recording of the image information by the image recording device, whereby the medical consumable being held above the image recording device for recording by the operator, the recording being initiated electronically and/or manually by the operator or is automatically initiated at regular intervals to provide the image information.

4. Method according to claim 1, wherein the method further comprises:
conducting of speech synthesis in order to provide an artificial generation of a human speaking voice from the audio information in order to acoustically issue the audio information via the audio device based on the human speaking voice.

5. Method according to claim 1, wherein the method further comprises:
provision of an interface for communication with a merchandise management system and/or a patient data management system,
transmitting at least the partial classification information and/or information resulting therefrom to the merchandise management system and/or to the patient data management system.

6. The method according to claim 5, wherein the method further comprises:
transmission of at least one identification characteristic of the patient, through the patient data management system to the image evaluation means, in order to subsequently add at least the one identification characteristic to the audio information in order to verify at least the one medical consumable to the correct patient.

7. Method according to claim 1, wherein the image evaluation means is designed as a neural network.

8. Method according to claim 1, wherein the method further comprises:
conducting of a training of the image evaluation means,
whereby the image evaluation means is trained and validated using training data, the training data being used for training and the remaining proportion of the training data being used for validation, whereby the training data contain at least one piece of image information of at least one medical consumable.

9. Method according to claim 8, wherein the conducting of the training further comprises:
acquiring a training image information,
conducting a detection of an image of at least the one medical consumable in the acquired training image information, whereby the acquired training image information is used as training data if the detection indicates a presence of the image, and otherwise the acquired training image information is used as a reference for the training.

10. Method according to claim 8, wherein the performance of the training also comprises:
acquiring a confirmation of the classification information of the image evaluation means based on the output of the audio information via the audio device by an operator.

11. Method according to claim 8, wherein the training data comprises at least one piece of training image information and a brightness of at least the one piece of training image information varies in order to enable the application of the image evaluation means when the brightness of the at least one piece of image information varies.

12. Method according to claim 8, wherein the training data comprises at least one piece of training image information in which the medical consumable to be acquired is partially obscured.

13. Method according to claim 8, wherein the training data includes at least one piece of training image information in which at least two medical consumables to be recorded are present.

14. Method according to claim 8, wherein the method further comprises:
making available an external database and an external data processing device in order to perform the image evaluation.

15. Method according to claim 14, wherein the external database which provides at least the one classification information of at least the one medical consumable, whereby the classification information of at least the one medical consumable is assigned to at least one form.

16. Method according to claim 14, wherein the procedure further comprises:
transmitting the respective image information acquired by the image recording device to the external data processing device in order to subsequently determine the classification information of at least the one medical consumable present in the external database and associated with the acquired image information, whereby the image information is thereby specific for one form of the medical consumable in that the image information comprises an image of the shape of the medical consumable in order to subsequently use the classification information corresponding to the shape to generate the audio information if the image information assigned to the respective classification information in the external database correlates with the imaged shape in the range of a specified order of magnitude.

17. Method according to claim 14, wherein the method further comprises:
updating the database, whereby the updating being carried out regularly by transmitting training data, the training data being created by:
conducting of a training of the image evaluation means, whereby the image evaluation means is trained and validated using training data, the training data being used for training and the remaining proportion of the training data being used for validation, whereby the training data contain at least one piece of image information of at least one medical consumable.

18. Method according to claim 14, wherein the method further comprises:
a storage of at least the one recorded medical consumable assigned to a medical condition and/or a patient in order to enable individual recording of required medical consumables per patient and/or per medical condition.

19. Method according to claim 8, wherein the classification information comprises a designation of the medical consumable in order to enable a check as to whether at least the one medical consumable has been correctly recorded, whereby for the check an operator using the issued audio information compares via the audio device at least the one acquired medical consumable.

20. Method according to claim 8, wherein the classification information additionally comprises a batch number of at least the one medical consumable.

21. System for acquiring at least one medical consumable comprising:
an image recording device for providing at least one piece of image information about at least the one medical consumable, whereby the image information is specific for a form of the medical consumable,
an image processing device for conducting an application of an image evaluation means, with the provided image information as an input of the image evaluation means in order to use an output of the image evaluation means as classification information about the medical consumable,
an audio processing device for generating audio information based on the classification information,
an audio device to conduct an acoustic output of the audio information.

22. System according to claim 21, wherein the system is designed as a stand-alone system, whereby a single, common housing is provided for all components of the system.

23. System according to claim 21, wherein the image recording device features a camera, the image recording device having at least one floor element and/or frame in order to be set up in a room, in such a way that the camera is aligned in the direction of a ceiling of the room.

24. System according to claim 21, wherein the image recording device features at least one lamp, to illuminate a viewing area of the image recording device.

25. System according to claim 21, wherein the system features an interface for a data connection with a merchandise management system and/or a patient data management system, whereby the interface is arranged in the housing.

26. System according to claim 21, wherein the system has a water-repellent and/or water-impermeable coating to be designed for use in a medical environment.

27. System according to claim 21, wherein the system is designed to be carried out by:
provision of an image recording device,
performing a provision of image information about at least the one medical consumable by the image recording device, whereby the image information is specific to a shape of the medical consumable,
performing an application of an image evaluation means, for shape recognition of the shape of the medical consumable, with the provided image information as an input of the image evaluation means to use an output of the image analysing means as a classification information about the medical consumable,
generating audio information based on the classification information in order to initiate an acoustic output of the audio information via an audio device.

28. Computer programme, comprising instructions which, when the computer programme is executed by a computer, cause the computer to carry out the method according to claim 1.
